SEQUENCE LISTING (1 sequence)

SEQ ID NO: 1 - protein, 10 aa, Artificial Sequence
  Spermatic biostabilizer FB-sp
RPGLPVFSPL

(19) United States
(12) Patent Application Publication
(10) Pub. No.: US 2020/0045956 A1
(43) Pub. Date: Feb. 13, 2020

(54) POLYPEPTIDE SPERM STABILISER FOR SEMEN USED IN ARTIFICIAL INSEMINATION

(71) Applicants: UNIVERSIDAD DE LA FRONTERA, Temuco, Temuco (CL); SOCIEDAD AGRICOLA Y GANADERA PEHUEN LTDA., Victoria, Araucania (CL)

(72) Inventors: Fernando Gonzalo ROMERO MEJIA, Temuco, Temuco (CL); Ignacio Andres JOFRE FERNANDEZ, Temuco, Temuco (CL); Antonio DE MIRANDA, San Pablo (BR); Sergio Andres REYNE HEISE, Victoria, Araucania (CL)

(21) Appl. No.: 16/482,341

(22) PCT Filed: Jan. 31, 2017
(86) PCT No.: PCT/IB2017/050516

§ 371 Date: Aug. 21, 2019

(51) Int. Cl. A01N 1/02; A61D 19/02; C12N 5/076; C07K 7/06; C07K 7/08

(52) U.S. Cl. CPC A01N 1/0226; A61D 19/02; C07K 7/08; C07K 7/06; C12N 5/061

(57) ABSTRACT

A polypeptide sperm bio-stabiliser FB-sp having the following sequence of amino acids: RPGLPVFSPL is provided; the use of the polypeptide bio-stabiliser for preserving the gametes of animals; a composition having the polypeptide bio-stabiliser; a conventional or alternative method for artificial insemination involving the use of the composition in conjunction with extender means to be injected into the females of animal species for commercial use.

### FIELD OF THE INVENTION

The present invention describes a polypeptide molecule having pharmacodynamic property, herein termed as FB-sp, which acts as a sperm stabiliser to be added to extensor media forming a mammal semen stabilising solution for mammals such as porcine, caprine, bovine, wild boars, horses, canine, felines, and even humans, preserving and conserving the germplasm of their gametes under refrigeration during the time between obtaining the semen sample from a breeding male and the time when this is used in artificial insemination (AI) methods.

### BACKGROUND OF THE INVENTION

Demand for animal meat (protein) and its derivatives increases annually, so livestock breeding farms must increase their production to meet said growing demand.

Artificial insemination (AI) method is the assisted reproduction technique most widely used by farms and/or producers of animals of commercial interest.

The current artificial insemination (AI) market provides nutritious liquid media, named sperm diluents or extenders, which can be added with bio-stabiliser molecules of different quality and price used to maintain and preserve sperm obtained from breeding male seminal fluid for longer periods of time.

AI technique requires collecting the male semen and adding preservation media containing a composition of micronutrients (extenders) that allow to obtain a higher spermatozoa survival, using these media the samples are maintained and protected under controlled cold, in the case of porcine sperm, for example, samples are kept between 15° C.-17° C. before being used in the process of female artificially assisted insemination.

Thus, the animal breeding industry needs molecular components as additive in sperm diluents or extenders, which have the functional property of stabilising sperm viability in order to strengthen the sperm fertilizing potential in AI process, either used for a single or a backup (second dose) insemination dose in order to improve the “fertilizing potential” conditions of the spermatozoa in the development of effective doses for AI to enhance the number of live births.

Diluents or extenders have been classified into two large groups, those whose objective is short-term conservation (less than 3 days), and those whose objective is long-term conservation (from 3 a 5 days). The former are mainly used in structures for short-term distribution of seminal doses, typical of European or Latin American systems, where the production of seminal doses in the same farm is frequent. While the long-term are typical of structures such as those present in the United States of America or Norway, where the distance between the place of seminal obtainment and production and the place to be used, i.e. female livestock breeding farms, is long. The advantages of long-term diluents are the possibility of preserving sperm viability over time for long-distance transport.

Methods of quality control and sperm safety are associated with semen diagnostic tests which are performed before the sperm is used, such as tests using PCR (Polymerase Chain Reaction) techniques to detect the presence of several viruses or complete analysis of seminal quality, whereby task organization in seminal collection farms is better organized and makes much easier sample distribution to breeding farms. However, long-term media have costs that make their massive and routine use impractical for livestock breeding farms in the intensive animal farming, in addition to requiring an antibiotic overload to ensure maintaining sample safety.

Xenobiotic factors are induced or produced during semen manipulation, such as thermal and biochemical changes that cause oxidative stress, which affect spermatozoa by damaging cell membranes, inducing an acrosomal reaction, decreasing their fertilizing potential and viability. This causes significant economic losses because the number of fertilized gametes is reduced, and thus the number of live births and consequently the animal production process decreases.

For preventing the induction of oxidative stress in concentrated ejaculated semen, the market has generated products such as liquid media known as seminal fluid diluents or extenders, which allow to preserve sperm cell functional characteristics and maintain the fertility level in this medium, artificially increasing the diluted volume of ejaculate. Media (extenders) available on the market, which are supplied to maintain the high metabolic activity necessary for sperm transport through the female genital tract, have not yet been able to solve the problem of short- and long-term spermatozoa preservation until their use in animal AI and, additionally, to maintain and preserve the germplasm, metabolic and biochemical capacity provided by male testicular seminal plasma, since they are affected by dilution (osmotic shock) inducing a reduction of the spermatozoa's reproductive efficiency.

The breeding males are classified as good or bad breeders, so that their selection is vital for obtaining breeders that provide semen having a maximum fertilizing potential “Premium males”, which is critical for an intensive livestock farms. In order to preserve spermatozoa for extended periods, it is necessary to modulate the sperm metabolic activity by reducing temperature, so one of the functional objectives of sperm diluents is to refrigerate and preserve ejaculates having high phenotypic quality features to allow the progeny to inherit meat quality and the genotypic features of the selected male breeders belonging to Premium category, which are identified by having high rates of cell viability, sperm motility, low morphological aberration content and high fertilization rate at reproductive level.

Traditional diluents or extenders have a capacity for preserving spermatozoa's shell life from 5 to 7 days, and are designed for balanced osmotic levels and pH, at values from 7 to 8. However, their bioprotective capacity is still relatively low since they show a viability drop greater than 40% in the first 24 hours. Long-term diluents allow preserve spermatozoa with viability rates between 40 and 65% while short-term diluents reach only up to 45% at third day. These products would be useful for transporting the sample from livestock breeding farms to other locations, keeping viability for several hours after obtaining the ejaculate, by keeping the storage temperature at 15° C., however, this is expensive.

The advantage of adding the FB-sp described in the present invention is to obtain a low-cost extender, which manages to maintain semen fertility and prolificacy better than commercial extenders without additives, achieving a survival of over 65% after 7 days, being useful for artificial insemination with fresh chilled spermatozoa or also extending its biochemical potential in the management of sperm cryopreservation at temperatures greater than −180° C.

WO2009043128 describes the effect proteases isolated from Bauhinia sp. for the treatment of microbial infections and pharmaceutical composition of a native sequence of more than 250 amino acids corresponding to an antibiotic product with bactericidal activity. On the contrary, the present invention focuses on a cell membrane stabiliser that uses absolutely different biological mechanisms.

In this context, the present invention discloses a polypeptide bio-protector spermatic stabiliser (FB-sp), which is an inexpensive biotechnology polypeptide molecular alternative, (short chain having 10 amino acids) for supplementation of spermatic diluents, allowing to extend the sample shell life while maintaining seminal quality parameters, for example, viability, metabolism, progressive sperm motility, fertilizing potential, ability to reduce oxidizing agents without interfering with the sperm physiology and metabolic capacity. This represents a greater impact on the reproductive and economic potential to massify any animal species, for example the massification of AI Premium males samples in farms distant from the origin sample sites or transnational shipments for genetic improvement.

### SUMMARY

The proposed invention describes a polypeptide molecule having pharmacodynamic property, of short chain of 10 amino acids, low molecular weight, which acts as a spermatic stabiliser to be added to extensor media for mammal semen, for example porcine, caprine, bovine, wild boars, horses, canine, felines, or even humans, allowing to preserve and conserve the germplasm of their gametes under refrigerated condition at −180° C. up to 17° C. for 60 hours with 75% to 95% fertility, the time between the collection of the semen sample of the reproductive male ejaculate and the time in which it is finally used in artificial insemination can be 0, 24, 36 or 48 hrs.

### BRIEF DESCRIPTION OF THE FIGURES

FIG. 1: FIG. 1 shows Progressive Gametes viability (%) vs. Time (hours) (●=Using FB-sp bio-stabiliser; ▪=without FB-sp bio-stabiliser), it shows percentage of pig spermatozoa with normal mobility suitable for fertilization, incubated in the presence of FB-sp 10 μM at 17° C.

FIG. 2: Concentration (ug/mL) of FB-sp in Plasma bbki4 vs. Time (hours). It shows plasma FB-sp concentration curve used in a liquid medium for a 90 kg animal.

### DETAILED DESCRIPTION OF THE INVENTION

The present invention describes a spermatic stabiliser, herein named FB-sp having an amino acid sequence, using a three letter nomenclature description, corresponding to: SEQ ID NO 1,
Arg, Pro, Gly, Leu, Pro, Val, Phe, Ser, Pro, Leu,

also written as one letter nomenclature:
RPGLPVFSPL,

An embodiment of the present invention is the use of said FB-sp additive to preserve male mammalian gametes, as well as the use of said additive to prepare a mammalian gamete bio-stabiliser composition for mammals such as porcine, caprine, bovine, wild boars, horses, canine, felines, or humans.

Other embodiment of the present invention is a bio-stabiliser composition comprising said FB-sp spermatic stabiliser used as a lyophilized product, at a concentration of up to 10 μM (10 micromol), diluted in any extender medium available in the state of the art, the concentration of the FB-sp may increase up to 10% for treating low-ratio living cells.

Other embodiment is the previous bio-stabiliser composition optionally added with any cryopreservant available in the state of the art.

Other embodiment of the present invention consists of a process for preserving mammalian semen comprising the steps of:

a) Separating by centrifugation, at a value from 35 to 45 g for 5 to 15 minutes, the cell fraction (spermatozoa) of the semen obtained by manipulation of breeding males from the liquid fraction consisting of nutrient liquid medium, which allows settling and separating living cells from inert cell debris that is removed from the process;

b) Diluting the living cells in physiological medium, wherein short- or long-term extenders are added to form a bio-stabiliser composition;

c) Adding the sperm stabiliser FB-sp of sequence SEQ ID NO: 1 at a concentration of up to 10 uM to the bio-stabiliser composition of step b) to form a fertilizing solution;

d) Keeping the fertilizer solution of stage c) in a cryopreservant medium at a temperature in the range from −180° C. to 17° C., stored in an insulated capped flask, without contact with the environment until use, the fertilizing solution can be stored or transported under these conditions, optionally the temperature range is from 1° C. to 17° C.;

e) Transporting the fertilizing solution to the place in situ and raising its temperature to 17° C. at the time of use and applying the fertilizing solution in the breeding females according to the artificial insemination protocols known in the state of the art;

f) Optionally in step c) a reactive oxygen species (ROS) stabilising complement is additionally added to the fertilizing solution when the living cell ratio is very small (<20% relative to the average pattern obtained for boards of the porcine farm). The stabiliser supplement may be the same FB-sp spermatic stabiliser increased by 10% in concentration, or the addition of traditional commercial antioxidant molecules such as albumins and others. The concentration will depend on the indications of each antioxidant molecule, considering that xenobiotic molecules that induce a hyperosmolarity shock (greater than 300 mOSM) should never be added;

### APPLICATION EXAMPLE

### Example 1

A test of the progressive motility of gametes incubated in fertilizing solution added with the FB-sp bio-stabiliser polypeptide was performed. Progressive gametes are spermatozoa that have normal mobility capacity and are suitable for fertilizing.

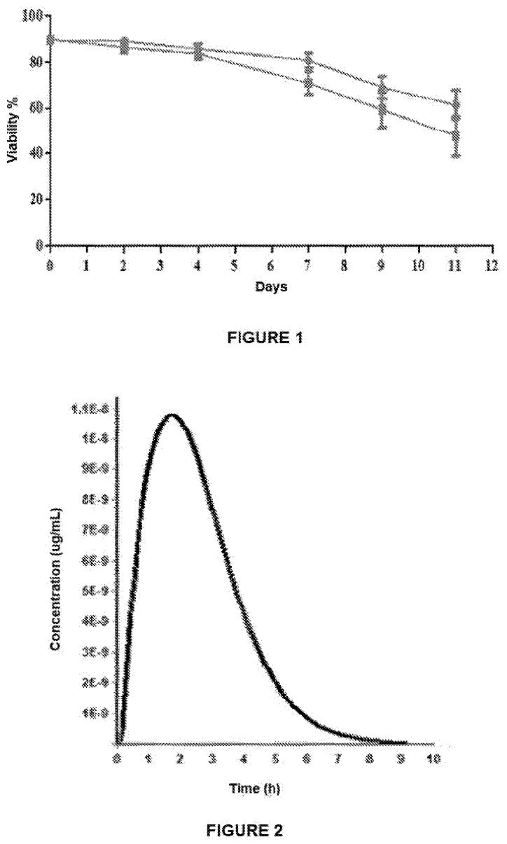

FIG. 1 shows the Progressive Gametes viability (%) percentage vs. Time (hours) (●=using FB-sp bio-stabiliser; ▪=without FB-sp bio-stabiliser)

The results show the protective effect of FB-sp on the progressive motility of porcine spermatozoa stored for 72 h at 17° C. It can be seen that the incubation of spermatozoa of porcine boars with FB-sp (●) (10 μM) preserves progressive motility up to 85% of spermatozoa for a period of 7 days, while the control (▪) without stabiliser decreases to a value of 70% on day 7, the difference increases over the days. These results are measured through optical microscopy, as measured by the CASA method for pig spermatozoa evaluation. This result is highly significant in relation to the values obtained when only spermatozoa incubation physiological media known in the art are used, such as MR-A® or Androstar®.

It is thus found that the FB-sp bio-stabiliser polypeptide provides a spermatozoa protective effect by extending their viability.

An analysis “in silico” made using GastroPlus simulation software provide some features of the FB-sp bio-stabiliser polypeptide: 1279 Da molecular weight, the structure of the 10 amino acids of FB-sp shows that only the conservative amino acid V (valine) and R (arginine) have conditions of active sites, i.e., the external pole Ac− has a negative charge and the external pole —NH2 (terminal amino acid), is positive, which determines that it is a hydrophilic and polar structure.

It was also seen that for a dose of 1 mg/mL, the absorption coefficient is 0.079 and the physicochemical dissolution coefficient in aqueous medium is 9.93×10³, depending on the calibration of the equipment in ionic liquid media used as controls, MR-A® medium was used in this case. This means that it has a low absorption capacity in biological membranes such as spermatozoa membranes, vaginal mucosa membranes, intrauterine mucosa membrane, but it has a very good dissolution capacity in physiological medium thus forming an extensor solution.

FIG. 2 shows the plasma concentration curve free of product FB-sp (μg/mL) administered in a liquid medium. When simulating the process for an animal weighing 90 kg, it can be seen that its blood plasma, after using the fertilizing solution in a concentration of 1 mg/mL of FB-sp diluted in MR-A® nutrient medium (nutritive medium for the preservation of porcine semen), has concentrations after 8 hours on the order of only 1.1×10⁻⁸ μg/mL in plasma and that its maximum pick is obtained after 2 hours. Then, the “in silica” model shows that it has a very low permeability coefficient and low residual effect because it is fully degraded from the organism within the first 8 hours.

Demonstrating that the applied concentration does not have a cytotoxicity risk.

1. A spermatic bio-stabiliser polypeptide FB-sp, comprising an amino acid sequence according to SEQ ID NO: 1.

2. The stabilising polypeptide of claim 1, wherein it serves to preserve mammalian gametes.

3. The stabilising polypeptide of claim 1, wherein it serves to prepare a bio-stabiliser composition to preserve mammalian gametes.

4. The stabilising polypeptide of claim 2, wherein mammals are selected from the group consisting of porcine, caprine, bovine, wild boars, horses, canine, felines.

5. The stabilising polypeptide of claim 2, wherein the mammal is human.

6. A sperm bio-stabiliser composition, further comprising:
the FB-sp spermatic bio-stabiliser of claim 1 in a concentration of up to 10 μM diluted in an extender medium.

7. The composition of claim 6, further comprising a cryopreservant medium.

8. A process to stabilise animal semen, wherein the semen obtained from the ejaculate undergoes a manipulation process that includes the following stages:
a) separating the cell fraction (sperm) of the semen, obtained by manipulation of the breeding males, from the liquid fraction consisting of nutrient liquid medium;
b) diluting the living cells in physiological medium, wherein short- or long-term extenders are added to form a bio-stabiliser composition;
c) adding the FB-sp spermatic stabiliser of claim 1, in the bio-stabiliser composition obtained in step b), in a concentration of up to 10 μM, to form a fertilizing solution;
d) keeping the fertilizing solution obtained in step c) in a cryopreserved or cold medium, at a temperature in the range from −180° C. to 17° C., stored in an insulated capped flask without contact with the environment until use;
e) the fertilizing solution can be stored or transported under these conditions; and
e) transporting the fertilizing solution to the place in situ, and raising the temperature to 17° C. and applying the fertilizing solution in the females at the time of use, according to the artificial insemination protocols known in the art.

9. The process according to claim 8, wherein in step d), the temperature range is between 1° C. and 17° C.

10. The process according to claim 8, wherein in step a), the breeding males are selected from the group consisting of porcine, caprine, bovine, wild boars, horses, canine and felines.

11. The process according to claim 8, wherein in step a), the breeding males are human.

12. The process according to claim 8, wherein in step c), a reactive oxygen species (ROS) stabilising complement at a concentration of from 0.001-100 μM is added to the fertilizing solution.
